(19) United States
(12) Patent Application Publication
(10) Pub. No.: US 2024/0090788 A1
(43) Pub. Date: Mar. 21, 2024

(54) SYSTEM AND METHOD FOR USING BLOOD FLOW MEASUREMENTS TO DETERMINE VENTRICULAR CONTRACTILITY

(71) Applicant: Guy P. Curtis, San Diego, CA (US)

(72) Inventor: Guy P. Curtis, San Diego, CA (US)

(21) Appl. No.: 18/161,049

(22) Filed: Jan. 28, 2023

Related U.S. Application Data
(60) Provisional application No. 63/375,906, filed on Sep. 16, 2022.

(51) Int. Cl. A61B 5/029; A61B 5/0205; A61B 5/1455

(52) U.S. Cl. CPC A61B 5/029; A61B 5/0205; A61B 5/14551

(57) ABSTRACT

The present invention pertains to a system and method for evaluating the blood volume-flow waveform of a patient for the purpose of determining ventricular contractibility. Input data for this evaluation includes measurements of oxygen saturation levels (SpO₂) in the waveform and a time duration for a respective cardiac cycle. Specifically, the oxygen saturation level SpO₂ in a waveform is indicative of an arterial blood-flow volume “V”. The data processor of a computer is then used for calculating a maximum rate of change in the arterial blood-flow volume “V” per time dV/dt. In accordance with the present invention, the maximum dV/dt for a succession of cardiac cycles are then compared for a clinical evaluation of trends in a patient's ventricular contractibility.

### FIELD OF THE INVENTION

The present invention pertains to systems and methods for monitoring ventricular contractibility. In particular, the present invention pertains to systems and methods for measuring a blood volume-flow waveform in the vasculature of a patient and for evaluating changes in the waveform for the stated purpose of monitoring ventricular contractibility. More particularly, the present invention is directed to systems and methods that use the waveform to calculate a maximum rate of change in arterial blood flow volume “V” per time, dV/dt, for a clinical evaluation of trends in ventricular contractibility.

### BACKGROUND OF THE INVENTION

A blood volume-flow waveform has many measurable characteristics that can be detected by an oximeter. These characteristics include oxygen saturation levels (SpO₂) in the blood, the time duration of cardiac cycles, and the time rate of change, dV/dt, in the blood flow volume-flow during a cardiac cycle. Moreover, it is known that the amount of red and IR light from an oximeter which is being absorbed during a cardiac cycle is indicative of fluctuations in oxygen saturation level SpO₂ in the blood. Further, it is also known that the magnitude of blood volume-flow will correspondingly fluctuate with SpO₂. Of particular importance here is the fact that these fluctuations in the magnitude of blood volume-flow can be detected by an oximeter and presented as a blood volume-flow waveform.

It is well known that the onset of cardiac systole in a cardiac cycle is denoted by the onset of the cycle's QRS complex. Furthermore, it is also known that the onset of an increase in arterial blood volume-flow occurs simultaneously with this onset of the cardiac cycle's QRS complex. It is therefore appropriate to measure dV/dt immediately after the QRS complex. Furthermore, it is also appropriate to use this dV/dt measurement as an indicator of the patient's heart muscle ventricular contractibility.

With the above in mind, it is an object of the present invention to provide a system and method for monitoring a patient's ventricular contractability that measures changes in blood volume-flow using an oximeter. Another object of the present invention is to use a blood volume-flow waveform generated with the measurements taken by the oximeter to monitor trends in ventricular contractability in a clinical/surgical environment. Yet another object of the present invention is to provide a system and method for monitoring a patient's ventricular contractability that is easy to manufacture, is simple to operate and is comparatively cost effective.

### SUMMARY OF THE INVENTION

Blood flow through the vasculature of a patient is caused by ventricular contractions of a patient's heart muscle that pump blood from the heart muscle into the vasculature. Specifically, in a cardiac cycle, the heart muscle will experience systole when the heart muscle contracts, and it will experience diastole when the heart muscle relaxes between contractions. In the vasculature, this pumping action results in a blood volume wave form that is indicative of the actual blood volume being pumped during each cardiac cycle.

It is well known that the onset of cardiac systole in a cardiac cycle is denoted by the onset of a QRS complex in the cycle. Further, it is known that the onset of an increase in arterial blood volume “V” occurs simultaneously with this onset of the cardiac cycle. Thus, the value for a time rate of change in blood volume “V” that is being, dV/dt, is an indicator of the efficacy of ventricular contractions of the heart muscle.

In the arteries of a patient, where blood flow volume is more dynamic, blood flow can be characterized by a central compartment where blood flow is relatively constant, i.e. dV/dt is steady. This compartment is generally referred to as the DC compartment. On the other hand, during an increase in blood flow volume in the artery which is caused by the pumping action of the heart muscle, dV/dt in the arterial volume peripherally surrounding the DC compartment will change cyclically. Moreover, it happens that dV/dt is significant. This compartment is generally referred to as the AC compartment.

During a cardiac cycle there are three variables involving an interaction between the light emitted by an oximeter and the reaction of a patient's arterial blood flow to the emitted light. These interactive variables are measurable during a cardiac cycle, and are:
1. An absorption rate, dA/dt, which gives the rate at which light emitted from an oximeter is absorbed in the patient's blood flow during the cardiac cycle;
2. The oxygen saturation level, SpO₂, that is achieved in the patient's blood during the cardiac cycle; and
3. The blood flow volume relative to the oxygen saturation level, SpO₂, which indicates a patient's ventricular contractibility for pumping blood through the patient's vasculature.

Light absorption rates, dA/dt, are determined by the wavelength of the light that is emitted from the oximeter. For purposes of the present invention, it is necessary for the oximeter to emit both a beam of red light and a beam of infrared light which have different wavelengths and different absorption rates. Respectively these absorption rates are, dA_{red}/dt, and dA_{IR}/dt. Typically, these different rates are evaluated as a modulation ratio “R” where R=[dA_{red}/dt]/[dA_{IR}/dt].

As noted above, an artery of a patient can be considered as two concentric tubular compartments. The central, inner compartment is non-pulsatile and has a constant volume. This is the DC compartment and is associated with d_{IR}/dt. On the other hand, the peripheral, outer compartment is pulsatile and has a changing volume during each cardiac cycle. This is the AC compartment, and it is associated with dA_{red}/dt.

Although It happens that both dA_{red}/dt, and dA_{IR}/dt fluctuate during a cardiac cycle, when “R” is considered as a double-ratio, [dA_{red}/dt]/[dA_{IR}/dt], the denominator dA_{IR}/dt has less influence on changes in the modulation ratio “R”. On the other hand, the numerator dA_{red}/dt which is associated with the AC compartment of blood flow is more transient and has a much more direct influence on changes in the modulation ratio “R”. Thus, absorbed light in the blood volume waveform rises and falls with the systole and diastole of the cardiac cycle due to respective increases and decreases in the pulsatile AC compartment blood volume waveform.

It has been empirically determined that at low values of SpO₂, the absorption amplitudes of red and infrared light in the AC compartment of an artery have a relationship resulting in a high value for the modulation ratio “R”. Conversely, at high values of SpO₂ this relationship results in a lower “R” value. A conclusion here is that because “R” is an inverse function of blood flow volume, an increased “R” results because of a lower arterial blood flow volume “V” with a lower dV/dt.

The consequence of changes in value for “R” is that an oximeter can measure “R” and the computer can use this information as an indicator of blood flow volume. In turn, blood flow volume “V” is indicative of ventricular conductivity. Hence, monitoring changes in dV/dt can be used to determine the efficacy of a heart muscle function.

### DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a view of a patient with an oximeter attached for an operation of the present invention;

FIG. 2 is a schematic presentation of functional compartments of the present invention and their interactive relationships;

FIG. 3 is a depiction of a blood volume flow waveform showing the relative volumes at a cross-section of an artery for the non-pulsatile (“DC”) and the pulsatile (“AC”) tissue compartments of the artery;

FIG. 4 shows red and infrared light absorbance characteristics of a blood volume flow waveform through a succession of cardiac cycles;

FIG. 5 shows changes in a red/infrared modulation ratio as a function of oxygen saturation SpO₂;

FIG. 6 is a view of an artery showing the effect an heart pumping function has on a cross-section of the between systole and diastole; and

FIG. 7 shows a variation in blood flow volume V during a cardiac cycle to emphasize the total contribution of this variation being from the AC compartment.

### DESCRIPTION OF THE PREFERRED EMBODIMENTS

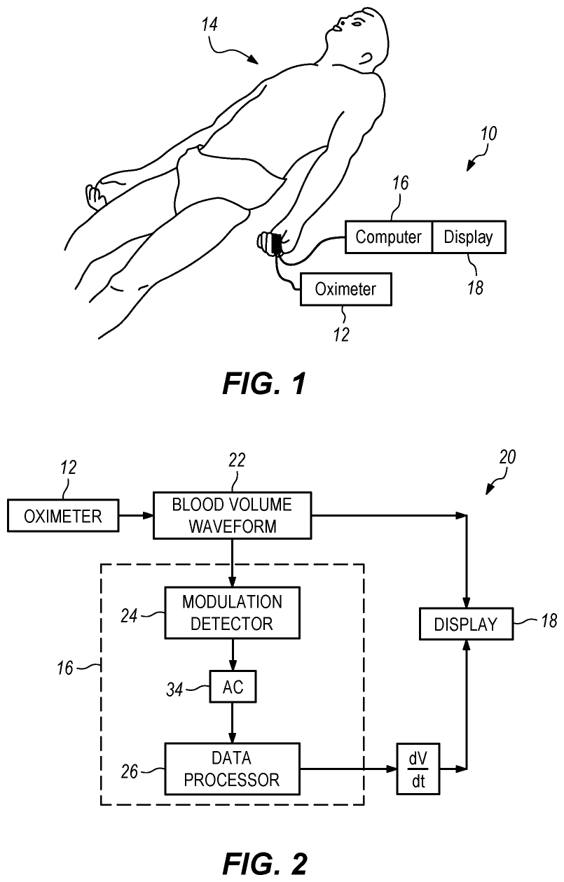

Referring initially to FIG. 1, a system for monitoring ventricular contractability is shown and is generally designated 10. As shown in FIG. 1, the system 10 essentially includes an oximeter 12 which is affixed to a patient 14 in a manner well known in the pertinent art. Also included in the system 10 is a computer 16 which is connected with the oximeter 12 and a display 18 which, in turn, is connected with the computer 16. For purposes of the present invention, the oximeter 12 is preferably of a type that transmits both a beam of red light and a beam of infrared light.

FIG. 2 is a schematic diagram 20 of system 10. As indicated in FIG. 2, oximeter 12 records a blood volume waveform 22 which monitors the volume of blood flow for the patient 14. Measurable characteristics of this blood volume waveform 22 is then transmitted to the display 18 and to a modulation detector 24 in the computer 16. Also shown is a data processor 26 which is incorporated into the computer 16.

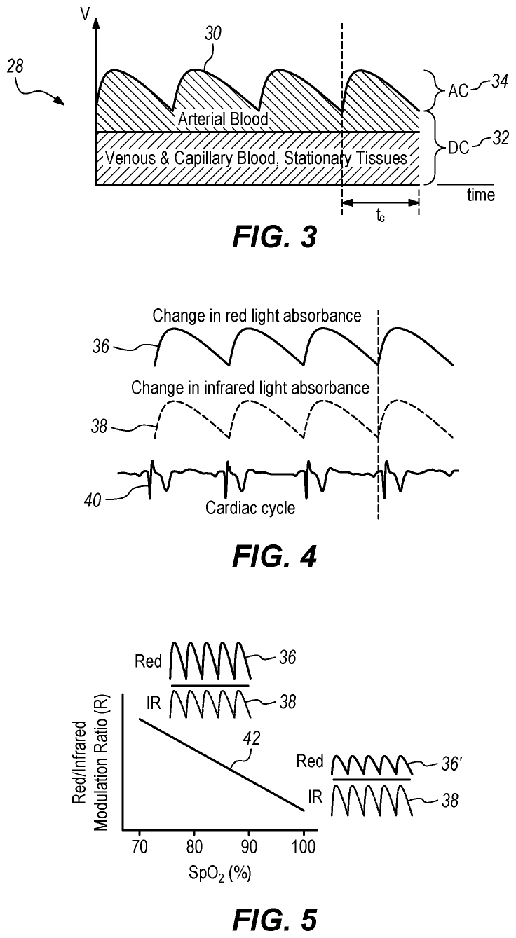

FIG. 3 is a graph 28 of a blood volume waveform 22 shows a plurality of successive pulses 30 as a function of pulse amplitude “A” with changes in time. In the graph 28 changes in the amplitude “A” over time is shown to indicate a change in the volume “V” of blood flow as a function of time dV/dt. Further, FIG. 3 shows that each pulse 30 occurs during a cardiac cycle with a time duration t c and that each pulse is characterized by changes in the volume “V”. Specifically, each pulse 30 will include a non-pulsatile “direct current” (DC) compartment 32, and a pulsatile “alternating current” (AC) compartment 34. In the computer 16, the DC compartment 32 is differentiated from the AC compartment 34 by the modulation detector 24.

The physical differences between the AC compartment and the DC compartment are important for an operation of the system 10. Specifically, as shown in FIG. 3, the volume “V” of blood passing each location in an artery will differ during a cardiac cycle t c as a function of time. This blood volume is cumulative from the DC compartment 32 and the AC compartment 34. Therefore, the pulsatile nature of the blood volume waveform 22, dV/dt, is attributable to the measurable aspect of blood volume flow in the AC compartment 34. This aspect results from the volume-dependent light absorption rate in the respective DC/AC compartments 32 and 34 as a function of the blood's oxygen saturation level SpO₂.

In a preferred embodiment of system 10 for the present invention, the oximeter 12 will emit both a beam of red light and a beam of infrared light. In this combination, the emission of red light from the oximeter 12 will have a measured amplitude A_{red} which is associated with the AC compartment of the blood volume waveform 22 and a volume-dependent, absorption time rate of change, dA_{red}/dt. It happens over a cardiac cycle t_{c} that dA_{red}/dt is sensitive to the oxygen saturation level SpO₂ and will change accordingly. On the other hand, the emission of infrared light from the oximeter 12 will have a measured amplitude A_{IR} which is associated with the DC compartment of the blood volume waveform 22. In comparison with dA_{red}/dt, the time rate of change, dA_{IR}/dt, is less sensitive to the oxygen saturation level SpO₂ than dA_{red}/dt and is thus relatively constant. Accordingly, a modulation ratio R=[dA_{red}/dt]/[dA_{IR}] can be used as a predictable indicator of blood volume flow.

FIG. 4 shows a solid line curve 36 for changes in red light absorbance during a succession of cardiac cycles t c in a blood volume waveform 22. Likewise, a dashed line curve 38 shows contemporaneous changes in infrared light absorbance during the cardiac cycles t c of the same blood volume waveform 22. As shown, the curves 36 and 38 represent pulses 30 which are initiated by the QRS complex 40 of a heart muscle function cycle. As is well known in the pertinent art, the QRS complex 40 is the beginning of systolic in the cardiac cycle t c when the heart muscle begins with a contraction of the ventricle of a patient's heart muscle.

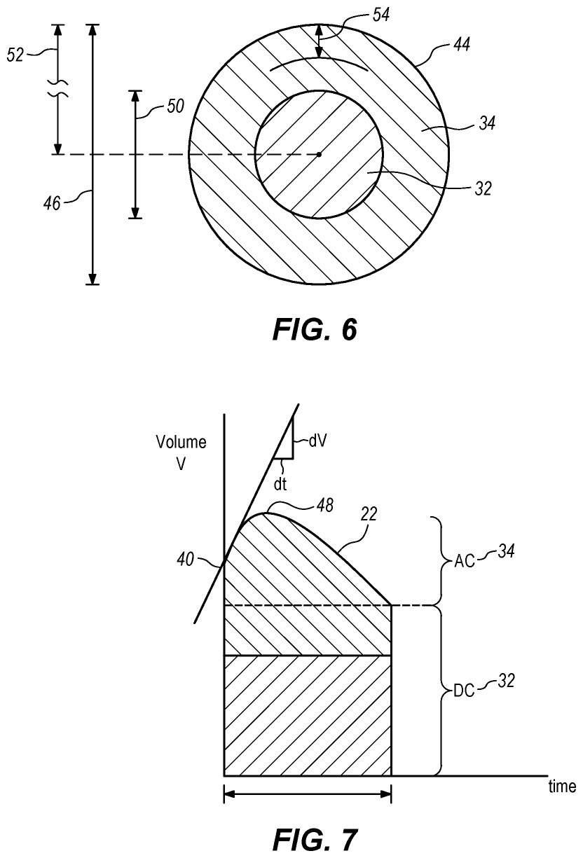

In FIG. 5 a line graph 42 showing changes in the modulation ratio R=[dA_{red}/dt]/[dA_{IR}] with changes in the oxygen saturation level SpO₂. Specifically, the numerator 36 of the modulation ratio “R” is shown to diminish to a value 36′, while the denominator remains relatively constant, as the oxygen saturation level SpO₂ increases. However, the oxygen saturation level SpO₂ increases due to an increase in volume dV/dt. And, an increase in volume follows from an increase in ventricular contractability.

FIG. 6 is a cross section of an artery 44 with a diameter of 46 of the artery at its maximum during a cardiac cycle t_{c}. At this point, FIG. 7 shows the apex 48 of the blood volume waveform 22. FIG. 7 also shows that the change in volume dV/dt will be most pronounced near the QRS complex 40 of the cardiac cycle t_{c}.

It is well known that during a cardiac cycle the cross section configuration of an artery 44 will change between a diameter 50 for the DC compartment 32 of a pulse 30, which is relatively constant, and a diameter 46 for the pulsatile AC compartment 34 of the pulse 30. More specifically, the radius 52 of the artery 44 will change from the apex 48 to a minimum by a radius change Δr 54. Blood volume flow dV/t will also change accordingly.

While the particular System and Method for Using Blood Flow Measurements to Determine Ventricular Contractility as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for monitoring ventricular contractibility which comprises:
a pulse oximeter attached to a patient for monitoring a blood volume flow waveform of the patient during a pulse of the patient's heart muscle cardiac cycle;
a computer connected to the pulse oximeter for receiving input data pertinent to the blood volume flow waveform, wherein the input data includes measurements of oxygen saturation levels (SpO₂) and a time duration for a cardiac cycle, wherein the oxygen saturation level SpO₂ is indicative of an arterial blood flow volume “V”;
a data processor in the computer for calculating a maximum rate of change in arterial blood flow volume “V” per time dV/dt, wherein dV/dt is based on measurements of the oxygen saturation levels SPO₂ in the blood volume flow; and
a display for presenting the maximum dV/dt in each cardiac cycle for clinical evaluation of trends in ventricular contractibility.

2. The system of claim 1 wherein the blood volume flow waveform comprises:
a non-pulsatile “direct current” (DC) compartment; and
a pulsatile “alternating current” (AC) compartment wherein the AC compartment is differentiated from the DC compartment by a modulation detector in the computer.

3. The system of claim 2 wherein the modulation detector evaluates a modulation ratio between the DC compartment and the AC compartment in the blood relative to changes in these compartments according to the oxygen saturation level SpO₂ in the blood.

4. The system of claim 3 wherein a predetermined range for the oxygen saturation level SpO₂ is above 92%.

5. The system of claim 2 wherein the pulse oximeter further comprises:
a first emitter for emitting red light, wherein the red light has a measured amplitude A_{red}, and wherein A_{red} is associated with the AC compartment of the blood flow waveform with a time rate of change, dA_{red}/dt, that is volume dependent; and
a second emitter for emitting infrared light, wherein the infrared light has a measured amplitude A_{IR}, and wherein A_{IR} is associated with the DC compartment of the blood flow waveform with a time rate of change, dA_{IR}/dt, that is relatively constant.

6. The system of claim 5 further comprising: a modulation detector in the computer for comparatively measuring an amplitude differential between the amplitude of red light absorption A_{red} relative to the amplitude of infrared light absorption A_{IR} during a cardiac cycle to establish therewith a modulation ratio R=A_{(red/IR)}.

7. The system of claim 6 wherein a time rate of change for the modulation ratio d R/dt is a ratio with a numerator [d R/dA_{red}]/dt and with a denominator [d R/dA_{IR}]/dt, and wherein the numerator [d R/dA_{red}]/dt is relatively changeable and the denominator [dR/dA_{IR}]/dt is relatively constant during a cardiac cycle.

8. The system of claim 7 wherein the modulation ratio “R” has a time rate of change dR/dt to provide a determinative measure of oxygen saturation SpO₂ as an indicator of blood flow volume dV/dt.

9. The system of claim 8 wherein the amplitude differential is a difference between a measure of red light absorption, A_{red}, and a measure of infrared light absorption A_{IR} during a cardiac cycle, wherein the total light absorption for oxygen saturation SpO₂ is equal to A_{red}+A_{IR}, where SpO₂ will fluctuate inversely with changes in the blood volume flow “V” between successive cardiac cycles as “V” increases/decreases and “R” decreases/increases.

10. The system of claim 9 where the time rate of change for the modulation ratio dR/dt is measured immediately following the QRS complex of a cardiac cycle.

11. A method for monitoring the ventricular contractability of a patient's heart muscle which comprises the steps of:
using an oximeter for measuring cyclical changes in characteristics of a blood flow waveform in an artery of the patient, wherein the measured characteristics are changes in an oxygen saturation level, SpO₂, with consequent changes in a modulation of the blood flow waveform;
evaluating time rate changes in light absorption levels dA/dt in the blood flow with changes in SpO₂ as evidence of waveform modulations resulting from diametrical variations of the artery; and
identifying diametrical variations in the artery as being indicative of volumetric flow variations resulting from the efficacy of the patient's ventricular contractability.

12. The method of claim 11 wherein the blood volume flow waveform comprises a non-pulsatile “direct current” (DC) compartment and a pulsatile “alternating current” (AC) and wherein the method further comprises the steps of:
differentiating the AC compartment from the DC compartment;
emitting red light from the oximeter for use in the evaluating step, wherein the red light has a measured amplitude A_{red}, and wherein A_{red} is associated with the AC compartment of the blood flow waveform with a time rate of change, dA_{red}/dt, which is volume dependent; and
emitting infrared light from the oximeter for use in the evaluating step, wherein the infrared light has a measured amplitude A_{IR}, and wherein A_{IR} is associated with the DC compartment of the blood flow waveform with a time rate of change, dA_{IR}/dt, that is relatively constant.

13. The method of claim 12 further comprising the step of comparing an amplitude differential between the amplitude of red light absorption A_{red} relative to the amplitude of infrared light absorption A_{IR} during a cardiac cycle to establish therewith a modulation ratio R=A_{(red/IR)}.

14. The method of claim 13 wherein a time rate of change for the modulation ratio dR/dt is a ratio with a numerator [dR/dA_{red}]/dt and with a denominator [dR/dA_{IR}]/dt, and wherein the numerator [dR/dA_{red}]/dt is relatively changeable and the denominator [dR/dA_{IR}]/dt is relatively constant during a cardiac cycle.

15. The method of claim 14 wherein the amplitude differential is a difference between a measure of red light absorption, A_{red}, and a measure of infrared light absorption A_{IR} during a cardiac cycle, wherein the total light absorption A_{red}+A_{IR} for oxygen saturation SpO₂ will fluctuate inversely with changes in the blood volume flow “V” between successive cardiac cycles as “V” increases/decreases and “R” decreases/increases.

16. The method of claim 15 where the time rate of change for the modulation ratio dR/dt is measured immediately following the QRS complex of a cardiac cycle.
